(19) United States
(12) Patent Application Publication
(10) Pub. No.: US 2023/0110589 A1
(43) Pub. Date: Apr. 13, 2023

(54) APPARATUS AND METHOD FOR PROVIDING PATIENT ADMINISTERED AND PHYSICIAN PRESCRIBED AND MONITORED DRUG TITRATION

(71) Applicants: PETER ELLENBY, PORTLAND, OR (US); JORDAN DOMINIC, PORTLAND, OR (US); CHRISTOPHER DOMINIC, PORTLAND, OR (US); THOMAS WILLIAM ELLENBY, SAN JOSE, CA (US)

(72) Inventors: PETER ELLENBY, PORTLAND, OR (US); JORDAN DOMINIC, PORTLAND, OR (US); CHRISTOPHER DOMINIC, PORTLAND, OR (US); THOMAS WILLIAM ELLENBY, SAN JOSE, CA (US)

(21) Appl. No.: 17/952,307

(22) Filed: Sep. 25, 2022

Related U.S. Application Data
(63) Continuation of application No. 17/065,447, filed on Oct. 7, 2020, now abandoned.
(60) Provisional application No. 62/913,642, filed on Oct. 10, 2019.

(51) Int. Cl. G16H 20/10; G06F 21/31

(52) U.S. Cl. CPC G16H 20/10; G06F 21/31

(57) ABSTRACT

The invention is concerned with providing safe methods for users of prescription drugs to safely cease using the drug(s) without experiencing painful or dangerous withdrawal symptoms. For example, many patients consume benzodiazepines and opioids for long periods of time and the need exists for patients to taper off their usage. According to the present invention, an apparatus and method teaches allowing the user to taper and titrate off of the drug where the user is administered tapered doses of the drug by medical professionals, physicians, nurses etc. Therefore, the patient may assist with the process of reducing and then eliminating their use of certain drugs under the ultimate control of a responsible medical professional. Such a process avoids patient embarrassment associated with addiction and can promote faster recovery times. Such a process is associated with appropriate tracking and control software so that medical professionals maintain requisite control over their patients.

### PRIORITY CLAIMS

This application claims the benefit of U.S. application Ser. No. 17/065,447, filed on Oct. 7, 2020, which claims the benefit of U.S. Provisional Application Ser. No. 62/913,642, filed on Oct. 10, 2019, the contents of which are incorporated herein by reference.

### FIELD OF THE INVENTION

The invention generally relates to the titration of therapy regimens. In particular, the invention provides methods of titrating physician prescribed and monitored drugs, which are patient administered.

### BACKGROUND OF THE INVENTION

The use of multiple medications and/or treatment modalities in the treatment of individual patients is an increasingly commonplace occurrence. The elderly population and those suffering from acute illness including from pandemic or epidemic conditions are continuing to consume more and more drugs. In the United States and other developed nations, the pace of new drug development, from drug discovery to drug production, has accelerated greatly, and single diseases are now treated with multiple 20 drugs targeting different biochemical pathways or different aspects in the pathophysiology of a disease.

Dose titration with single compounds is a relatively straightforward process employed by physicians to identify appropriate dose levels which produce improved responses in patients while simultaneously minimizing the adverse side 30 effects a patient may experience. After taking into account a patient's age, weight, and other factors specific to the patient, the physician will prescribe an initial dose which may be increased or decreased as needed, depending on how the patient responds. This titration continues until a favorable balance between the desired response and undesirable side effects is achieved.

The present invention seeks to optimize the titration process.

### SUMMARY OF THE INVENTION

The following system is concerned with providing safe methods for users of prescription drugs to safely cease using the drug(s) without experiencing painful or dangerous withdrawal symptoms. Many people who have been taking prescription drugs for long periods of time, such as benzodiazepines and opioids, may not realize that if they wish to cease taking these drugs that they need to taper off of them. This is due to the fact that their bodies have become, often without the user being aware, physically addicted to the drug. Simply stopping the drug suddenly can have very adverse and dangerous consequences, including death.

The most commonly recommended method to allow the user to taper, or titrate, off of the drug is inpatient or outpatient rehabilitation, where the user is administered tapered doses of the drug by medical professionals such as physicians, or nurses etc. These are very costly programs and they also carry some degree of stigma and embarrassment while undergoing treatment, not to mention the patient may need to miss a significant time from work or other matters. Another problem with traditional methods is that many people do not live close to a suitable facility, greatly limiting their ability to stop using an undesired prescription.

The present invention is an apparatus and method for Patient Administered and Physician Monitored Methods of Drug Titration, consisting of a user interaction portal that will most likely be, but not limited to: an application (“app”) on a mobile device, such as a smart phone or a tablet; a website, along with the support of back end services including but not limited to the titration plan generation program; and, actual physical and mental health professionals who will be available to users via the portal while undergoing the process.

The patient is guided through the titration process with the assistance of a patient portal that may consist of an online, web based solution or a mobile application (“app”) that would run on a smart phone or other device. This portal provides everything needed for the patient to safely taper off of the prescription or substance. All patient activity will be logged by the system and patient data will be used and analyzed to inform and shape future patient titration schedules and processes. In one iteration, the portal would consist of four main components that provide services customized to the needs of each patient, as recommended by physicians and mental health experts.

The first component is the patient plan, which is a general overview of the titration process and access to their physician.

The second component is medication, where the patient can view their current dose level, progress and schedule, and also log and confirm their intake of the medication to inform the system of their progress. The medication component of the portal will also send reminders or alerts to the patient for when a scheduled dose is due for consumption.

The third component is the body, or physical health of the patient. While undergoing treatment, each user will have access to physicians and nurses via text, online chat or phone. The patients will be able to track any physical symptoms they are experience as they titrate. Physical activities and exercise will be encouraged and be logged by the patient. It may also be possible to monitor a patient's vitals, heart rate, oxygen levels, blood pressure, etc., via peripherals that connect to the portal via Bluetooth or other means.

The fourth component is the mind, or mental health of the patient. While undergoing treatment each client will have access to therapists and metal health experts via text, online chat or phone. There will be access to live group therapy sessions. Users will be able to record and share their stories about their addiction and their tapering process as well as listen to other patient's stories and these could be compiled into a “best of” podcast for patients to listen to. The portal may also incorporate therapeutic aspects such as meditation & relaxation exercises, and Eye Movement Desensitization and Reprocessing (EDMR), which is primarily used to overcome symptoms associated with post-traumatic stress disorder (PTSD) and has been found to effectively treat other mood and anxiety disorders, including depression, phobias, and panic.

While engaging with the portal, it may be that case that reported mental or physical patient data may alter the titration schedule. This may result in an increase, a speeding up of the process, or a decrease, a slowing of the process. This may be done automatically by the system or from a physician altering the plan based on patient submitted data. This would result in the system generating a new set of prescriptions for the updated titration schedule. Things that may cause an alteration the titration schedule may include, but are not limited to, the following; patient mood & personality; unreported long term use of a drug (e.g., at 1 month, 3 months, 6 months, 1 year, 18 months, 2 years, 3 years, etc.); panic & stress patient response; illness or physical problems; biometric data; sleep patterns; changes in heart rate or pulse rate; weight gain or loss; and data collected from smartwatch EKG technology or other wearable technology that can interface to invention.

In another embodiment, the present invention uses drugs crafted into pills that are precisely dosed for a user's needs. These pills may be made to order for each patient, or they may be manufactured in a manner in that almost each varying degree of dosing is represented. Each user, according to their current prescription, dosage amount and other factors, will have a titration plan created for them by the system. The plan will then be approved either by their referring physician or a system physician. If a user is referred to the system by a physician, the user's fitness must be verified by the referring physician in order to commence the process. If the user accesses the system without a referring physician, the system will determine if their case will allow safe use of the titration system.

Once a patient is verified, the system will create a titration plan for them. This titration plan will be generated based on an analysis of the drug, and the usage or dosage that the user is titrating from. The system may also analyze many factors, including but not limited to: the drug; the user's current dosage; the length of time that the user has been taking the drug; the user's weight and age; other drugs the user is taking; alcohol use; and, general health of the user. Verification of the patient is required to access to the portal. This can be done in a number of ways including, but not limited to: user name and password; biometric recognition of a fingerprint, face or an eye; scanning a barcode on a wristband provided to and worn by the patient; voice recognition; text verification and more.

Besides access via a variety of methods, chat, phone call or online group meetings, to physical and mental health professionals through the portal patients using the system will also have access to stress reducing and relaxation methods such as mediation and EMDR to assist them through the titration process. Access to all of these services may continue even after the titration process has been completed. Once the titration process is complete and the user is down to a zero, null dose, it may be that the portal is still available to the patient to log their physical and mental stages as they continue life without the substance and post-titration. This data will also be used to inform and shape future titration schedules and plans for future patients.

These and other aspects, objects, features and advantages of the present invention, are specifically set forth in, or will become apparent from, the following detailed description of an exemplary embodiment of the invention.

### BRIEF DESCRIPTION OF THE DRAWINGS

FIG. 1 is a flowchart showing a possible mode of operation of the system.

FIG. 2 is a flowchart showing a possible mode of operation of the titration dosage monitoring subsystem.

FIG. 3 is a flowchart showing a possible more advanced mode of operation of the system taking into account patient withdrawal symptoms, injury, or other patient reported drug use.

FIG. 4 is a flowchart showing a possible more advanced mode of operation of the system including individual physician authorized prescriptions for each individual different dose as may be required by some jurisdictions.

### DETAILED DESCRIPTION OF THE PREFERRED EMBODIMENT

In accordance with the preferred embodiments of the inventions, Patient Administered and Physician Monitored Methods of Drug Titration are provided. It will be appreciated that each of the embodiments described include methods and that the methods of one preferred embodiment may be different than the methods of another embodiment. The preferred embodiments described herein are not the sum total of the invention but merely contain certain methods of how the disclosed invention may provide unique experiences to users of the system.

The present invention is an apparatus and method for Patient Administered and Physician Monitored Methods of Drug Titration, consisting of a user interaction portal that will most likely be, but not limited to: an application (“app”) on a mobile device, such as a smart phone or a tablet; a website, along with the support of back end services including but not limited to the titration plan generation program; and, actual physical and mental health professionals who will be available to users via the portal while undergoing the process.

In one embodiment, the present invention uses drugs crafted into pills that are precisely dosed for a user's needs. These pills may be made to order for each patient, or they may be manufactured in a manner in that almost each varying degree of dosing is represented. Each user, according to their current prescription, dosage amount and other factors, will have a titration plan created for them by the system. The plan will then be approved either by their referring physician or a system physician. If a user is referred to the system by a physician, the user's fitness must be verified by the referring physician in order to commence the process. If the user accesses the system without a referring physician, the system will determine if their case will allow safe use of the titration system.

Once a patient is verified, the system will create a titration plan for them. This titration plan will be generated based on an analysis of the drug, and the usage or dosage that the user is titrating from. The system may also analyze many factors, including but not limited to: the drug; the user's current dosage; the length of time that the user has been taking the drug; the user's weight and age; other drugs the user is taking; alcohol use; and, general health of the user.

The following is a typical titration plan;
Week 1: This constitutes the beginning of the taper process. Little to no reduction occurs.
Week 2: Dose reduced by 25 percent.
Week 3: Dose is once again reduced by 25 percent.
Week 4: Total reduction of 50 percent by this point.
Week 5-8: No change in dose for one month.
Week 9 and Beyond: 25 percent reduction per week until cessation or desired dose reached.

For the first example of the system in use, we have a user who was prescribed 2 mg. of the benzodiazepine Alprazolam a day, dosed into four 0.5 mg. doses, after being diagnosed with cancer. The drug was prescribed to limit the stress and panic such a diagnosis can manifest in a patient. The patient is still taking the drug longer than necessary and it is beginning to alter the patient's mood and personality. Three years after the cancer diagnosis and three years after beating the disease they are still taking the drug and they want to stop. They inform their physician of this decision and the doctor refers them to the use of the titration system described in this disclosure instead of going to a rehabilitation facility. The doctor submits the patient information to the system, drug, dosage etc., and gives a system Rx Code to the user. The user then loads the user portal, in this case an app they install on their smart phone, and creates a user ID and password. Once logged in they enter their system Rx Code and the system accesses their drug usage information provided by the patient's doctor and generates a titration plan for the user.

Alprazolam titration plan;
Week 1, Normal dose 2 mg.*
Week 2, 1.5 mg.
Week 3, 1.13 mg.
Week 4, 1 mg.
Weeks 5-8, 1 mg.
Week 9, 0.75 mg.
Week 10, 0.5 mg.
Week 11, 0.25 mg
Week 12, 0 mg. *daily dose to be divided into four pills per day, e.g. Week 2 four 0.375 mg. pills per day

This consists of a calendar, dosage times and dosage amounts for the patient. This plan would be sent to their physician for approval. Once approved by their doctor, drugs are then regularly dispatched to the patient, either to their home or work, or a pharmacy for pick up. The drugs are tapered doses delivered in order of strongest to weakest to facilitate the tapering process. Once the drugs have arrived the user alerts the system and the titration process begins. The system will send reminders to the patient whenever a dose is scheduled, and the user will inform the system that they have taken their dose. These alerts to take the scheduled dose may come in a manner of ways including but not limited to app generated alerts, text or iMessages, emails and phone calls. This will progress throughout the titration process. If the user misses doses the system may automatically adjust the patient's plan to accommodate the missed doses. One can see that for the titration plan in this example it would be nearly impossible for a patient to undergo this precise dosing by themselves since cutting 1 mg., 0.5 mg. or 0.25 mg. pills into such precise small doses is not doable. While using the system, the patient is an integral part of the process since they are the ones administering and logging the prescribed titrated doses. It should be noted that a patient who misses doses early on in the titration process is more at risk due to the higher doses of the drug at this stage and doctor involvement would be triggered more quickly due to missed doses.

It is also possible that the patient begins the use of the titration system with a pre-prescribed titration plan from their referring physician. If this were the case the physician or the user could input the titration plan into the system and titration would proceed (if the user were to enter the plan information, this would require physician verification).

For a further example of the system in use we have a patient who has become reliant on a low dose of opioids after a minor surgery. They can't seem to stop taking the pills. They learn of the system as described herein and access the user portal via the website. They create a user log in and password. The system asks if they were referred and have an Rx Code. In this case they do not. The system then gathers the user's data to see if they may safely use the patient administered titration method provided by the system. This would, among other factors, take into account the user's potential additional psychological addiction to their prescription as well as the risk of them abusing the prescribed dosages. The patient has been taking two 5 mg. doses of opioids a day, 10 mg. total, for four months. It is not unusual for a patient to be discharged from a surgery with far more medication than needed and this is how the user became addicted to the opioids. Having entered their information, the system performs an analysis on the user data and determines that the user is a candidate for safe usage of the titration system. The system then generates a titration plan for the user.

Opioid titration plan;
Week 1, Normal dose 10 mg.*
Week 2, 7.5 mg.
Week 3, 5.63 mg.
Week 4, 4.22 mg.
Weeks 5-8, 4.22 mg.
Week 9, 3.47 mg.
Week 10, 2.11 mg.
Week 11, 1.06 mg
Week 12, 0 mg. *daily dose to be divided into two pills per day, e.g. Week 2 two 3.75 mg. pills per day

This plan is then reviewed by a system physician who decides to slightly modify the plan to 0.5 mg. for week 12 and then a drop to zero mg. for week 13. Once the titration plan is final the system physician issues an Rx Code to the patient and the titration process proceeds. While undergoing the process the user can track any physical or mental symptoms they are experiencing though the system portal and the system may adjust the user's titration plan based on these symptoms, any changes of course being approved by a physician.

One can imagine a multitude of further scenario where addicted patients utilize the system as described to safely taper off of a prescription.

It should be noted that the titration plans referenced in this disclosure are for example only and that actual titration plans along with the titration generation program and patient suitability factors to be able to use the system would be under the supervision and advice of medical professionals.

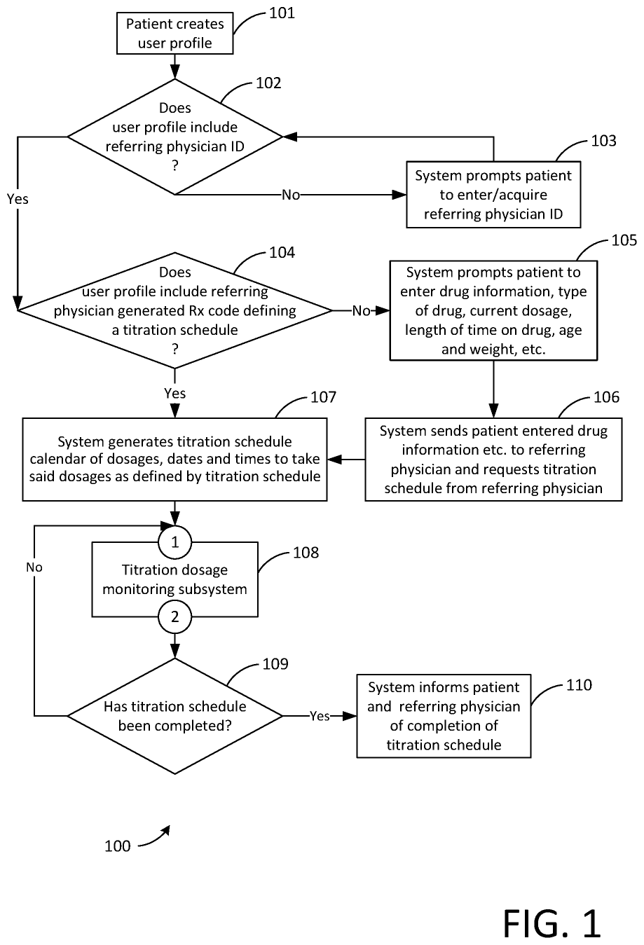

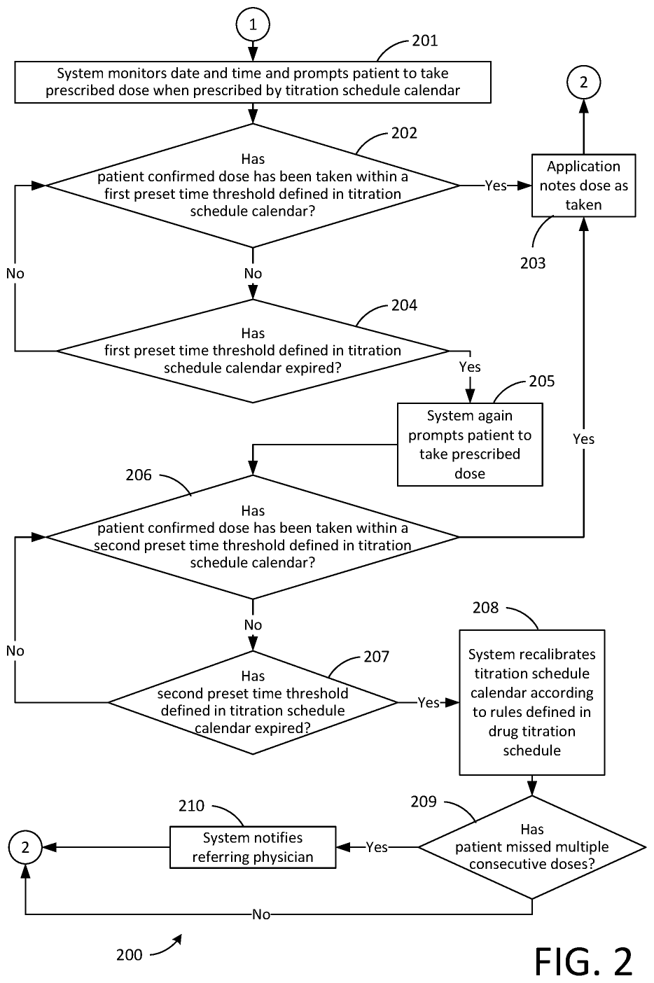

FIG. 1 is a flowchart 100 showing a possible mode of operation of the system. In step 101 the patient, the user of the system, logs into an existing user profile or creates one. The flowchart then branches to step 102. In step 102 the system determines if the user profile includes a referring physician ID. If the user profile does not include a referring physician ID the flowchart branches to step 103 in which the system prompts the patient to enter or acquire a referring physician ID. The flowchart then branches back to step 102. If the user profile does include a referring physician ID the flowchart branches to step 104. In step 104 the system determines if the user profile includes a referring physician generated Rx code defining a titration schedule. If the user profile does include a referring physician generated Rx code defining a titration schedule the flowchart branches to step 107. If the user profile does not include a referring physician generated Rx code defining a titration schedule the flowchart branches to step 105. In step 105 the system prompts the patient to enter the drug information (type of drug, current dosage and frequency, length of time on drug, etc.) age, weight, etc. The flowchart them branches to step 106. In step 106 the system sends the patient entered information re drug, etc. to the referring physician and requests a titration schedule from same. The flowchart then branches to step 107. In step 107 the system generates a titration schedule calendar of dosages, dates and times to take said dosages as defined by the titration schedule. The flowchart then branches to step 108 via connector 1. In step 108 the system runs the titration dosage monitoring subsystem, a possible mode of operation of which is described in FIG. 2 and associated text. The flowchart then branches to step 109 via connector 2. In step 109 the system determines if the titration schedule has been completed. If the titration schedule has not been completed the flowchart branches back to step 108 via connector 1. If the titration schedule has been completed the flowchart branches to step 110 in which the system informs the patient and the referring physician of completion of the titration schedule.

FIG. 2 is a flowchart 200 showing a possible mode of operation of the titration dosage monitoring subsystem. From connector 1 the flowchart branches to step 201. In step 201 the system monitors the current date and time and prompts the patient to take the prescribed dose when prescribed by the titration schedule calendar. The flowchart then branches to step 202. In step 202 the system determines if the patient has confirmed that the prescribed dose has been taken within a first preset time threshold, e.g. within 15 minutes of being prompted, defined by the titration schedule calendar. If the patient confirms that the dose has been taken within the time threshold the flowchart branches to step 203 in which the system records the dose as taken and then branches to connector 2. If the patient does not confirm that the dose has been taken within the time threshold the flowchart branches to step 204. In step 204 the system determines if the first preset time threshold has expired. If the first preset time threshold has not expired the flowchart branches back to step 202. If the first preset time threshold has expired the flowchart branches 205 in which the system again prompts the patient to take the prescribed dose. The flowchart then branches to step 206. In step 206 the system determines if the patient has confirmed that the prescribed dose has been taken within a second preset time threshold, e.g. within 15 minutes of being prompted the second time, defined by the titration schedule calendar. If the patient confirms that the dose has been taken within the time threshold the flowchart branches to step 203 in which the system records the dose as taken and then branches to connector 2. If the patient does not confirm that the dose has been taken within the time threshold the flowchart branches to step 207. In step 207 the system determines if the second preset time threshold has expired. If the second preset time threshold has not expired the flowchart branches back to step 206. If the second preset time threshold has expired the flowchart branches 208. In step 208 the system recalibrates the titration schedule calendar according to rules defined in the titration schedule. This recalibration may involve new prescriptions being generated by the system and sent to the referring physician for review and approval. The flowchart then branches to step 209. In step 209 the system determines if the patient has missed multiple consecutive doses. If the patient has not missed multiple consecutive doses the flowchart branches to connector 2. If the patient has missed multiple consecutive doses the flowchart branches to step 210 in which the system informs the referring physician of the multiple consecutive missed doses by the patient, i.e. the failure to comply with the titration schedule calendar. The flowchart then branches to connector 2.

Other factors that may alter a patient's titration plan may be illness or injury that occur during the process, travel, etc. All of these factors would be reported to the system via the user portal and the system, with the approval of either the patient's referring physician or a system physician, may alter the patient's titration plan.

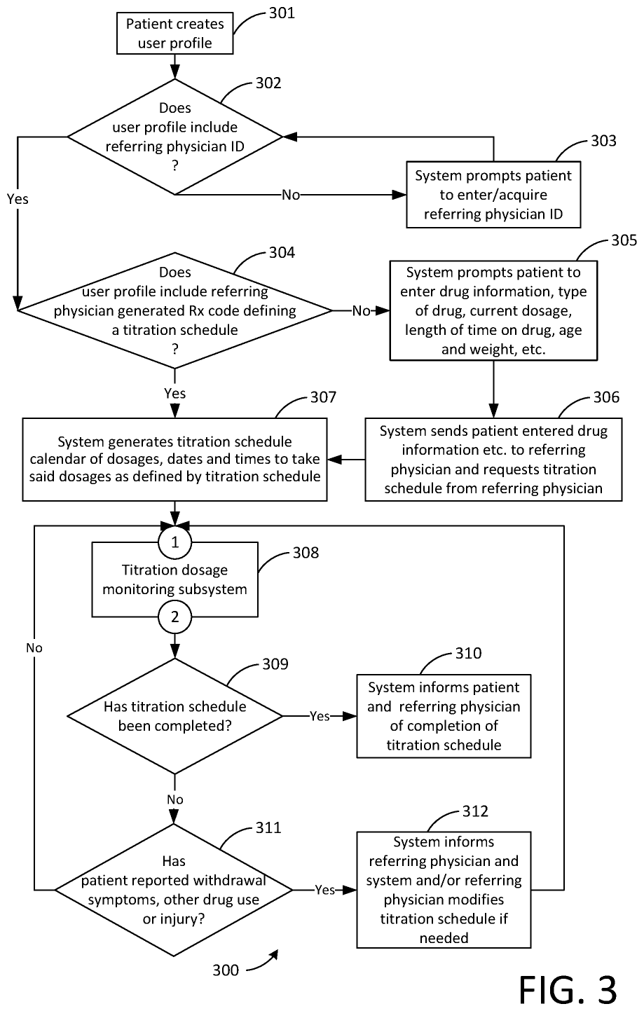

FIG. 3 is a flowchart 300 showing a possible more advanced mode of operation of the system taking into account patient withdrawal symptoms, injury and/or other patient reported drug use (i.e. the patient took an additional dose of the drug that the titration schedule is related to or the patient took a dose of another drug). In step 301 the patient, the user of the system, logs into an existing user profile or creates one. The flowchart then branches to step 302. In step 302 the system determines if the user profile includes a referring physician ID. If the user profile does not include a referring physician ID the flowchart branches to step 303 in which the system prompts the patient to enter or acquire a referring physician ID. The flowchart then branches back to step 302. If the user profile does include a referring physician ID the flowchart branches to step 304. In step 304 the system determines if the user profile includes a referring physician generated Rx code defining a titration schedule. If the user profile does include a referring physician generated Rx code defining a titration schedule the flowchart branches to step 307. If the user profile does not include a referring physician generated Rx code defining a titration schedule the flowchart branches to step 305. In step 305 the system prompts the patient to enter the drug information (type of drug, current dosage and frequency, length of time on drug, etc.) age, weight, etc. The flowchart them branches to step 306. In step 306 the system sends the patient entered information re drug, etc. to the referring physician and requests a titration schedule from same. The flowchart then branches to step 307. In step 307 the system generates a titration schedule calendar of dosages, dates and times to take said dosages as defined by the titration schedule. The flowchart then branches to step 308 via connector 1. In step 308 the system runs the titration dosage monitoring subsystem, a possible mode of operation of which is described in FIG. 2 and associated text. The flowchart then branches to step 309 via connector 2. In step 309 the system determines if the titration schedule has been completed. If the titration schedule has been completed the flowchart branches to step 310 in which the system informs the patient and the referring physician of completion of the titration schedule. If the titration schedule has not been completed the flowchart branches to step 311. In step 311 the system determines if the patient has reported any withdrawal symptoms, other drug use or injury. If the patient has not reported any withdrawal symptoms, other drug use or injury the flowchart branches back to step 308 via connector 1. If the patient has reported withdrawal symptoms, other drug use or injury the flowchart branches to step 312 in which the system informs the referring physician and the referring physician modifies the titration schedule if needed. The flowchart then branches back to step 308 via connector 1.

It may be the case that the patient resides in a geographic location that has laws requiring that any change in dosing requires a new prescription from a prescribing physician. If this were the case the system would generate the prescriptions, matching the unique doses of the drug titration regimen, for the physician to approve. These may all be approved in advance or as the patient progresses through the titration process. If any event occurred, such as missed doses or injury, that required a change to the titration dosing schedule the system would generate new prescriptions to match this change.

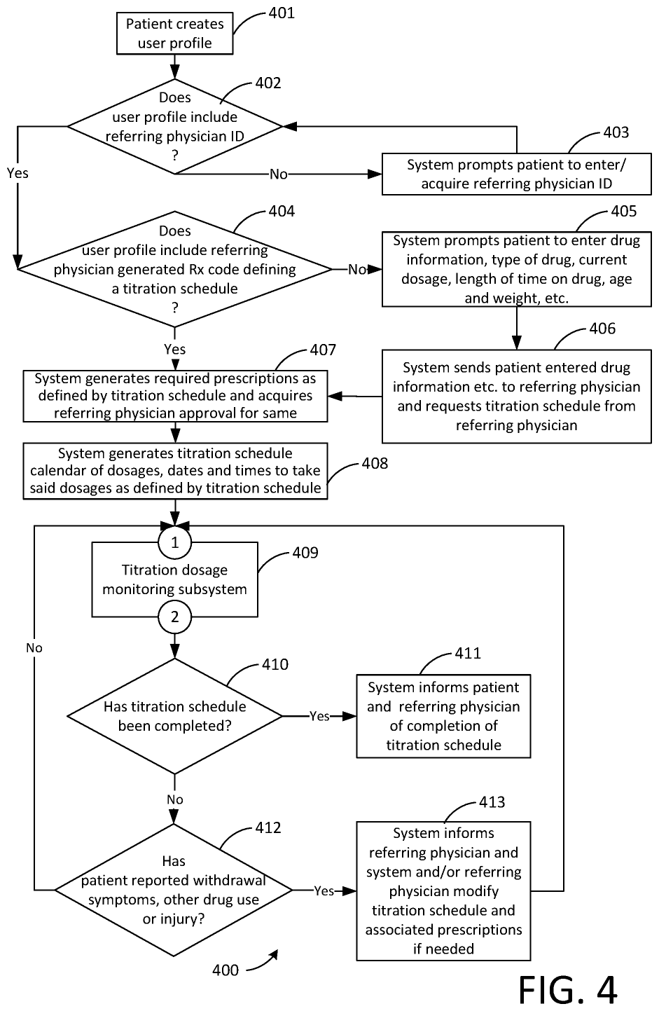

FIG. 4 is a flowchart 400 showing a possible more advanced mode of operation of the system including individual physician authorized prescriptions for each individual different dose as may be required by some jurisdictions. In step 401 the patient, the user of the system, logs into an existing user profile or creates one. The flowchart then branches to step 402. In step 402 the system determines if the user profile includes a referring physician ID. If the user profile does not include a referring physician ID the flowchart branches to step 403 in which the system prompts the patient to enter or acquire a referring physician ID. The flowchart then branches back to step 402. If the user profile does include a referring physician ID the flowchart branches to step 404. In step 404 the system determines if the user profile includes a referring physician generated Rx code defining a titration schedule. If the user profile does include a referring physician generated Rx code defining a titration schedule the flowchart branches to step 407. If the user profile does not include a referring physician generated Rx code defining a titration schedule the flowchart branches to step 405. In step 405 the system prompts the patient to enter the drug information (type of drug, current dosage and frequency, length of time on drug, etc.) age, weight, etc. The flowchart them branches to step 406. In step 406 the system sends the patient entered information re drug, etc. to the referring physician and requests a titration schedule from same. The flowchart then branches to step 407. In step 407 the system generates the required prescriptions as defined by the titration schedule and acquires the referring physician's approval for same. The flowchart then branches to step 408. In step 408 the system generates a titration schedule calendar of dosages, dates and times to take said dosages as defined by the titration schedule. The flowchart then branches to step 409 via connector 1. In step 409 the system runs the titration dosage monitoring subsystem, a possible mode of operation of which is described in FIG. 2 and associated text. The flowchart then branches to step 410 via connector 2. In step 410 the system determines if the titration schedule has been completed. If the titration schedule has been completed the flowchart branches to step 411 in which the system informs the patient and the referring physician of completion of the titration schedule. If the titration schedule has not been completed the flowchart branches to step 412. In step 412 the system determines if the patient has reported any withdrawal symptoms, other drug use or injury. If the patient has not reported any withdrawal symptoms, other drug use or injury the flowchart branches back to step 409 via connector 1. If the patient has reported withdrawal symptoms, other drug use or injury the flowchart branches to step 413 in which the system informs the referring physician and the referring physician modifies the titration schedule if needed. The system may also generate new prescriptions if needed and acquire the referring physician's approval for same. The flowchart then branches back to step 409 via connector 1.

Besides access via a variety of methods, chat, phone call or online group meetings, to physical and mental health professionals through the portal patients using the system will also have access to stress reducing and relaxation methods such as mediation and EMDR to assist them through the titration process. Access to all of these services may continue even after the titration process has been completed.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A method for titrating drugs for treatment of a disability including the following steps:
a. a drug prescriber issuing a drug prescription;
b. a patient desiring said drug prescription, wherein said drug prescription includes a schedule;
c. said patient takes said drugs in response to the continuation of said disability;
d. said patient continues to take said drugs beyond a time wherein the efficacy of said drugs is outweighed by a creation of a potential addiction to said drugs;
e. said patient initiates a request to said drug prescriber indicating a desire to discontinue use of said drug prescription;
f. said drug prescriber submits patient information to a patient drug database, inputting drug identification, drug dosage and recommended consumption intervals and provides a prescription code to said patient; and
g. patient then inputs said prescription code into said patient drug database and uses a patient-controlled application software-controlled communication device to access drug usage information including a titration plan to eliminate said drug usage by said patient.

2. A method according to claim 1 wherein said communication device is a smartphone.

3. A method according to claim 2 wherein a user of said smartphone must be authenticated by way of a secure username and password to insure that said titration plan is known only by said patient and said drug prescriber.

4. A method according to claim 1 wherein said titration plan includes dosage reductions over a period of time set my said drug prescriber in response to a request by said patient, and wherein said patient provides treatment success data to said drug prescriber so that said titration plan may be optimally modified.

5. A method according to claim 4 wherein said titration plan is stored within said patient drug database so that historical data relating to said patient is available for patient and second drug prescriber's usage in the future.

6. A system for titrating drugs for treatment of a disability including:
a. a drug prescriber issuing a drug prescription;
b. a patient desiring said drug prescription, wherein said drug prescription includes a schedule;
c. said patient takes said drugs in response to the continuation of said disability;
d. said patient continues to take said drugs beyond a time wherein the efficacy of said drugs is outweighed by a creation of a potential addiction to said drugs;
e. said patient initiates a request to said drug prescriber indicating a desire to discontinue use of said drug prescription;
f. said drug prescriber submits patient information to a patient drug database, inputting drug identification, drug dosage and recommended consumption intervals and provides a prescription code to said patient; and
g. patient then inputs said prescription code into said patient drug database and uses a patient-controlled application software-controlled communication device to access drug usage information including a titration plan to eliminate said drug usage by said patient.

7. A system according to claim 6 wherein said communication device is a smartphone.

8. A system according to claim 7 wherein a user of said smartphone must be authenticated by way of a secure username and password to insure that said titration plan is known only by said patient and said drug prescriber.

9. A system according to claim 6 wherein said titration plan includes dosage reductions over a period of time set my said drug prescriber in response to a request by said patient, and wherein said patient provides treatment success data to said drug prescriber so that said titration plan may be optimally modified.

10. A system according to claim 9 wherein said titration plan is stored within said patient drug database so that historical data relating to said patient is available for patient and second drug prescriber's usage in the future.

11. A method for titrating drugs for treatment of a disability including the following steps:
a. a drug prescriber issuing a drug prescription;
b. a patient desiring said drug prescription, wherein said drug prescription includes a schedule;
c. said patient takes said drugs in response to the continuation of said disability;
d. said patient continues to take said drugs beyond a time wherein the efficacy of said drugs is outweighed by a creation of a potential addiction to said drugs;
e. said patient initiates a request to said drug prescriber indicating a desire to discontinue use of said drug prescription;
f. said drug prescriber submits patient information to a patient drug database, inputting drug identification, drug dosage and recommended consumption intervals and provides a prescription code to said patient;
g. patient then inputs said prescription code into said patient drug database and uses a patient-controlled application software-controlled communication device to access drug usage information including a titration plan to eliminate said drug usage by said patient;
h. wherein said communication device is a smartphone;
i. data authentication installed within said smartphone including a secure username and password to insure that said titration plan is known only by said patient and said drug prescriber;
j. wherein said titration plan includes dosage reductions over a period of time set my said drug prescriber in response to a request by said patient, and wherein said patient provides treatment success data to said drug prescriber so that said titration plan may be optimally modified; and
k. wherein said titration plan is stored within said patient drug database so that historical data relating to said patient is available for patient and second drug prescriber's usage in the future.
